(19) United States
(12) Patent Application Publication
(10) Pub. No.: US 2011/0070631 A1
(43) Pub. Date: Mar. 24, 2011

(54) Container transportation case and culture treating device

(75) Inventor: Yasujiro Kiyota, Tokyo (JP)

(73) Assignee: NIKON CORPORATION, Tokyo (JP)

(21) Appl. No.: 12/923,388

(22) Filed: Sep. 17, 2010

Related U.S. Application Data
(63) Continuation of application No. PCT/JP2009/001185, filed on Mar. 17, 2009.

(30) Foreign Application Priority Data
Mar. 18, 2008 (JP) 2008-069211

(51) Int. Cl. C12M 3/00

(52) U.S. Cl. 435/286.1; 435/289.1

(57) ABSTRACT

A container transportation case is used to carry in/out a culture container to/from a chamber of a culture treating device. A case body of the container transportation case has a container housing room in which the culture container can be arranged and the container housing room is in communication with the outside by an opening part. A joint unit of the container transportation case can be attached/removed to/from a carrying-in/out opening of the culture treating device, and forms a space that is isolated from an outside atmosphere by joining the container housing room and the carrying-in/out opening. An airtight member of the container transportation case closes the opening part to block the container housing room from the outside atmosphere.

### CROSS REFERENCE TO RELATED APPLICATION

This application is a continuation application of International Application No. PCT/JP2009/001185, filed on Mar. 17, 2009, designating the U.S., in which the International Application claims a priority date of Mar. 18, 2008, based on prior filed Japanese Patent Application No. 2008-069211, the entire contents of which are incorporated herein by reference.

### BACKGROUND

1. Field

The present application relates to a container transportation case and a culture treating device.

2. Description of the Related Art

Conventionally, culture treating devices are known such as an incubator in which a living body sample is cultured in a chamber (temperature-controlled room or the like) maintained at a predetermined atmosphere and a clean bench to carry out a culture operation of a living body sample. Regarding such a culture treating device, as one example, Japanese Unexamined Patent Application Publication No. 2005-204546, a technique is also proposed that controls load and unload of the air in a culture treating device corresponding to opening and closing of a door in order to suppress flowing in/out of the air from a chamber when accessing from the outside.

### BRIEF DESCRIPTION OF THE DRAWINGS

FIG. 1 is a front view of a culture treating device system of a present embodiment.

FIG. 2 is a plan view of the culture treating device system of the present embodiment.

FIG. 3 is a block diagram of the culture treating device system of the present embodiment.

FIG. 4 is a side view of a container transportation case.

FIG. 5 is a side view of the container transportation case in an attaching state.

FIG. 6 is a plan view of the container transportation case.

FIG. 7 is a plan view of the container transportation case in an attaching state.

FIG. 8 is a rear view of the container transportation case in an attaching state.

FIG. 9 is a flowchart showing a behavioral example of carrying in/out of a culture container in the present embodiment.

### DETAILED DESCRIPTION OF THE EMBODIMENT

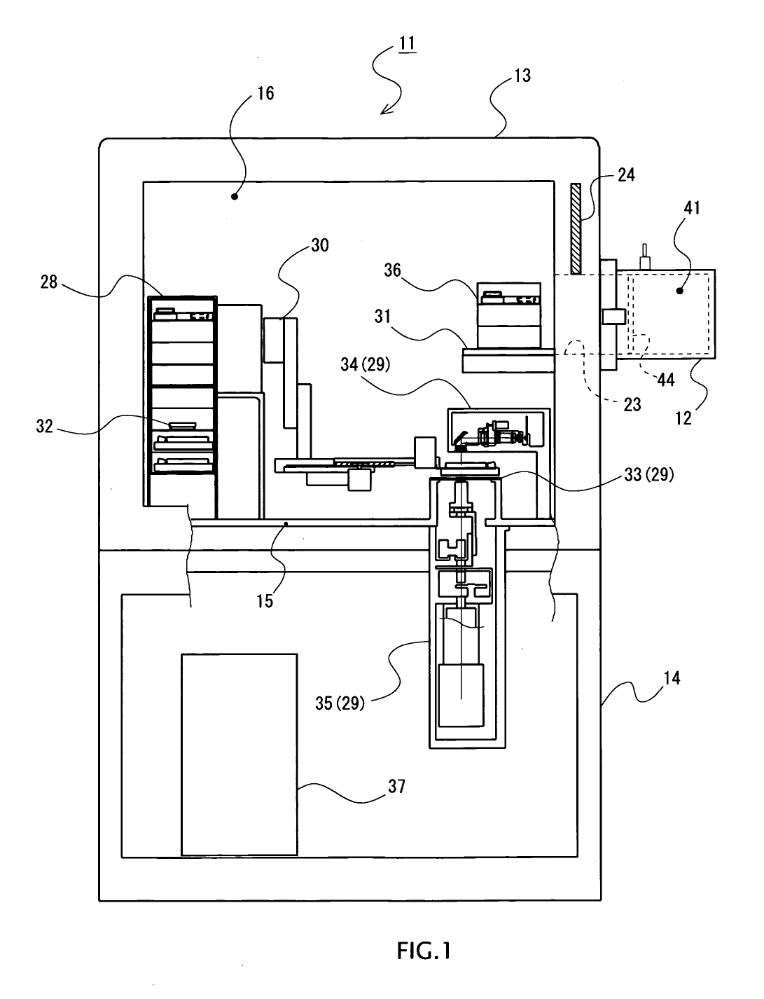

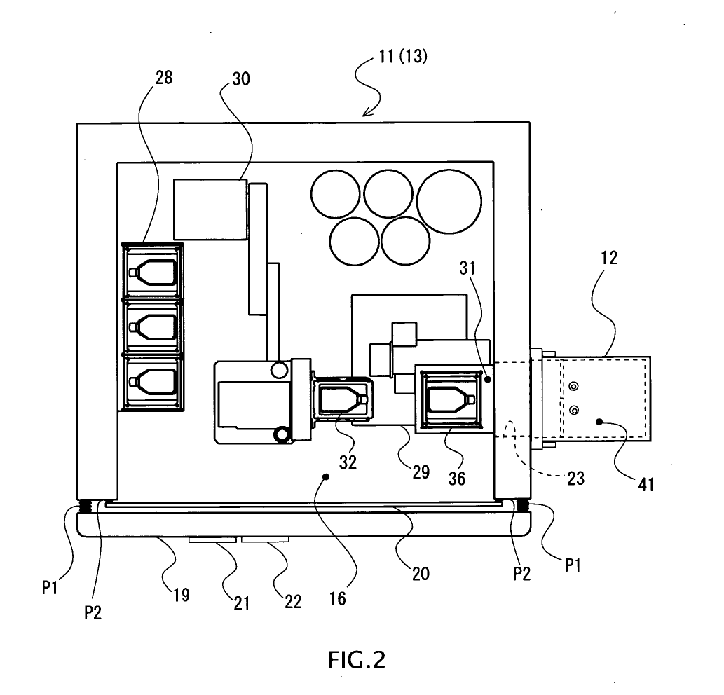

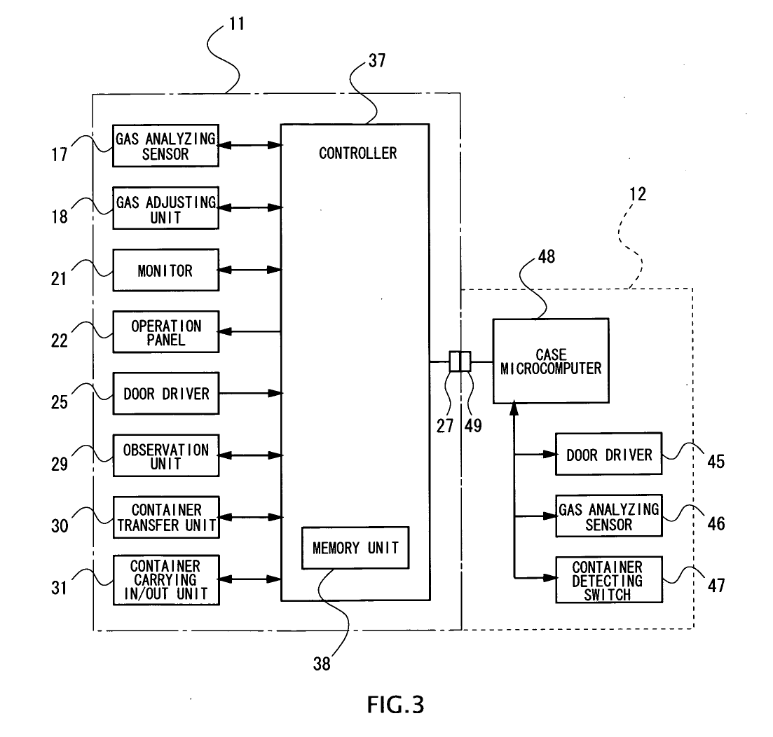

A description is given below of a configuration example of a culture treating device system in the present embodiment with reference to the drawings. The culture treating device system of the present embodiment has an incubator 11, which is one example of the culture treating device, and a container transportation case 12. FIGS. 1 and 2 are a front view and a plan view of the culture treating device system of the present embodiment. FIG. 3 is a block diagram of the culture treating device system of the present embodiment, respectively.

The incubator 11 of the present embodiment has an upper casing 13 and a lower casing 14. In an assembled state of the incubator 11, the upper casing 13 is placed on the lower casing 14. In addition, an internal space between the upper casing 13 and the lower casing 14 is vertically sectioned by a base plate 15.

Furthermore, inside the upper casing 13, a temperature-controlled room 16 is formed which carries out culture of a living body sample (a cell or the like). In the temperature-controlled room 16, a gas analyzing sensor 17 and a gas adjusting unit 18 are arranged. Meanwhile, in the temperature-controlled room 16, a temperature controller, a humidity controller, and the like may also be arranged further (illustration of the temperature controller, the humidity controller, and the like is omitted).

Here, the gas analyzing sensor 17 detects composition of an atmosphere gas in the temperature-controlled room 16. Further, the gas adjusting unit 18 is connected to a carbon dioxide tank, an oxygen tank, and a nitrogen tank (illustration of each tank is omitted). Then, the gas adjusting unit 18 introduces the gases from each tank into the temperature-controlled room 16 to adjust the composition of the atmosphere gas in the temperature-controlled room 16. This enables the incubator 11 to maintain the state in the temperature-controlled room 16 at a low oxygen environment suitable for culturing a living body sample (illustration of both the gas analyzing sensor 17 and the gas adjusting unit 18 is omitted in FIGS. 1 and 2). Meanwhile, the gas adjusting unit 18 has piping (not shown) which supplies a gas outside the temperature-controlled room 16 and can also supply the above gases to the container transportation case 12.

On a foreside (underside in FIG. 2) of the temperature-controlled room 16, an outer door 19 and an inner door 20 are arranged. The outer door 19 covers foresides of the upper casing 13 and the lower casing 14. The inner door 20 covers the foreside of the upper casing 13 inside the outer door 19 and isolates the environment of the temperature-controlled room 16 from the outside when the outer door 19 is opened. Moreover, the outer door 19 and the inner door 20 each have airtight properties maintained by packings P1 and P2. On the outer door 19, a monitor 21 and an operation panel 22 are mounted.

In addition, on the right side (the right in FIG. 2) of the temperature-controlled room 16, a carrying in/out opening 23 is opened which connects the temperature-controlled room 16 with the outside. The carrying in/out opening 23 is used when carrying in/out a culture container 32. Then, the carrying in/out opening 23 is sealed by an airtight door 24 that opens and closes by sliding. Meanwhile, the above airtight door 24 can be opened and closed automatically by a door driver 25.

In a surrounding area of the carrying in/out opening 23 outside the upper casing 13, an annular guide piece 26 that shows a attaching position of the container transportation case 12, and an electrical contact 27 to carry out communication with the container transportation case 12 are provided (the electrical contact 27 is shown only in FIG. 3).

In the temperature-controlled room 16, a stocker 28, an observation unit 29, a container transfer unit 30, and a container carrying in/out unit 31 are arranged.

The stocker 28 is arranged on the left-hand side of the temperature-controlled room 16 as viewed from the foreside (underside in FIG. 2) of the upper casing 13. The stocker 28 has a plurality of shelves, and each shelf of the stocker 28 can house a plurality of culture containers 32. Then, each culture container 32 accommodates a living body sample together with a culture medium.

The observation unit 29 is arranged on the right-hand side of the temperature-controlled room 16 viewed from the foreside of the upper casing 13. In the observation unit 29, time lapse observation of the living body sample in the culture container 32 can be performed.

The observation unit 29 is fitted into an opening part of the base plate 15 of the upper casing 13 for installation. The observation unit 29 has a sample table 33, a stand arm 34 hanging over the sample table 33, and a body portion 35 having a microscopic optical system and an imaging unit built in. Then, while the sample table 33 and the stand arm 34 are arranged in the temperature-controlled room 16, the body portion 35 is housed in the lower casing 14.

The sample table 33 is configured with a translucent material, and the culture container 32 can be placed thereon. The sample table 33 is configured movably horizontally and can adjust the position of the culture container 32 placed on the upper surface. The stand arm 34 has a built-in light source. Then, the imaging unit captures an image of the living body sample translucently illuminated from above the sample table 33 by the stand arm 34 via the microscopic optical system. Such configuration allows carrying out microscopic observation of a living body sample without changing environmental conditions.

The container transfer unit 30 is arranged at the center of the temperature-controlled room 16 as viewed from the foreside of the upper casing 13. The container transfer unit 30 is configured by mounting an arm to sandwich the culture container 32 at a tip end of a vertical robot having an articulated arm. This enables the container transfer unit 30 to carry out delivery and reception of the culture container 32 among the stocker 28, the sample table 33 of the observation unit 29, and the container carrying in/out unit 31.

The container carrying in/out unit 31 is arranged in the vicinity of the carrying in/out opening 23. In the example of the present embodiment, the container carrying in/out unit 31 is arranged above the observation unit 29. On the container carrying in/out unit 31, a rack 36 can be mounted which is capable of housing three culture containers 32 vertically. Then, the container carrying in/out unit 31 carries in/out the culture containers 32 including the rack 36 from the carrying in/out opening 23 by sliding the above holder to the outside of the carrying in/out opening 23. The putting in/out of the culture containers 32 to/from the rack 36 is carried out by the container transfer unit 30.

Meanwhile, in the lower casing 14, the body portion 35 of the observation unit 29 and a controller 37 are housed. Here, the controller 37 is a computer that carries out integrated control of the culture treating device system and is connected to the gas analyzing sensor 17, the gas adjusting unit 18, the door driver 25 of the airtight door 24, the monitor 21 and the operation panel 22, the observation unit 29, the container transfer unit 30, and the container carrying in/out unit 31, respectively (refer to FIG. 3). The controller 37 performs communication with a case microcomputer 48 on the side of the container transportation case 12 via the electrical contact 27. Further, the controller 37 has a built-in memory unit 38. In the memory unit 38, registration information of the culture container 32 housed in the temperature-controlled room 16, observation schedules of each culture container 32, data of microscopic images captured by the observation unit 29, and the like are memorized.

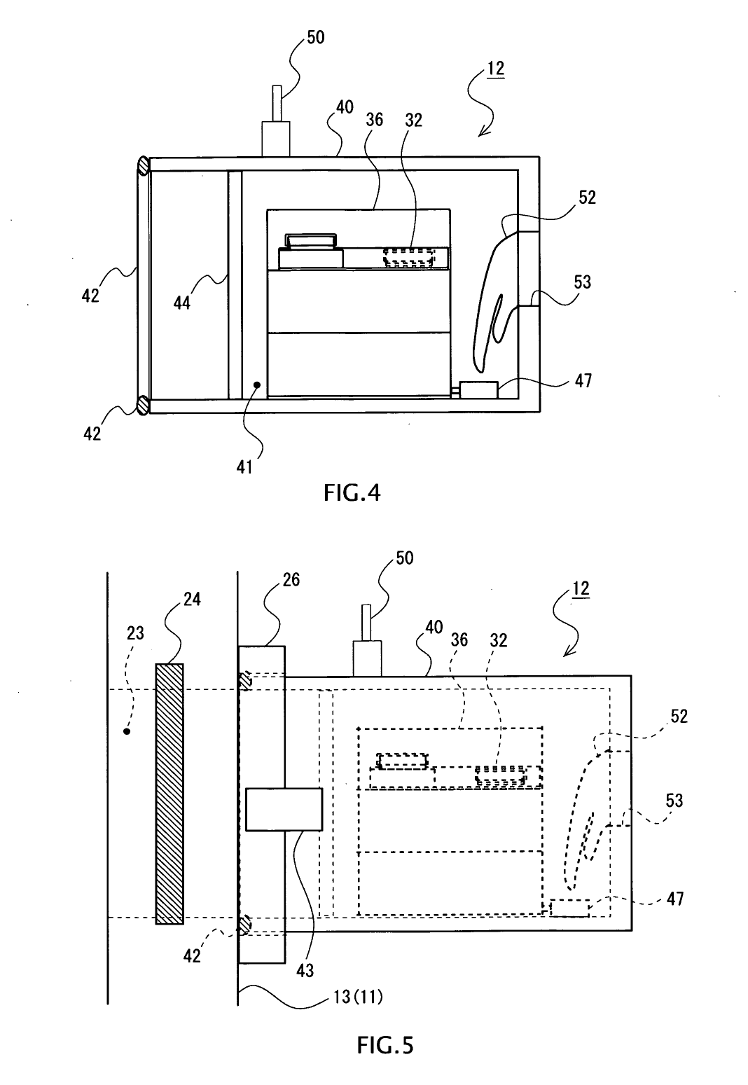

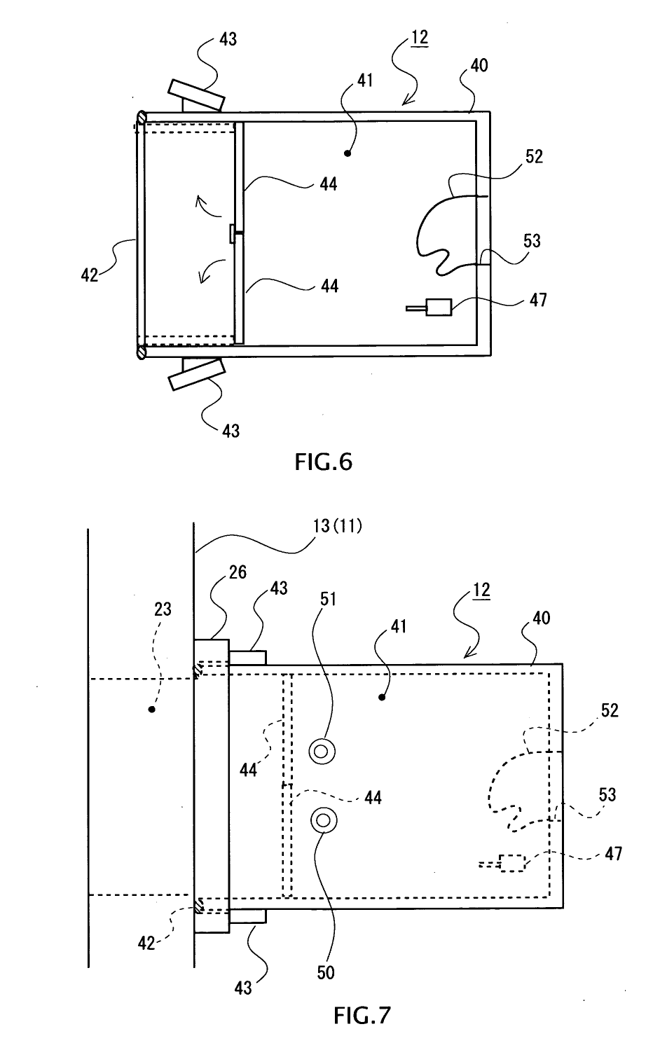

Next, with reference to FIGS. 4 through 8, a description will be given of a configuration of the container transportation case 12. The container transportation case 12 is configured to allow attachment outside the carrying in/out opening 23 of the incubator 11. Then, the container transportation case 12 is used when transporting the culture container 32 in a state of being isolated from an outside atmosphere or to carry in/out the culture container 32 in a state of being isolated from the outside atmosphere.

A case body 40 of the container transportation case 12 is formed in a rectangular parallelepiped shape and has a container housing room 41 that can accommodate the above rack 36 therein. One side (the right in FIGS. 4 through 7) of the case body 40 is opened in its entire surface so as to be in communication with the above container housing room 41. Further, a space from the container housing room 41 to the opening of the case body 40 is set to be in a size that allows the rack 36 to pass through. The case body 40 in the present embodiment is formed with a transparent material to allow visual recognition of a state in the container housing room 41.

The container transportation case 12 is attached to the incubator 11 in such a manner that the opening portion faces the carrying in/out opening 23. In the state of the container transportation case 12 being attached to the incubator 11, the case body 40 covers the carrying in/out opening 23 of the incubator 11 and also the opening part of the case body 40 tightly adheres to a wall of the incubator 11. At a front end of the case body 40, to maintain the air tightness in an attaching state of the container transportation case 12, an annular seal ring 42 is mounted.

Further, on both sides of the case body 40, fixtures 43 are provided respectively which engage to the guide piece 26 provided on the side of the incubator 11. Therefore, by engaging the fixtures 43 to the guide piece 26, the container transportation case 12 can be securely fixed to the incubator 11.

Further, inside the case body 40, a break-front partition door 44 is provided. The partition door 44 can be opened and closed by an electrically driven door driver 45 that is built in the container transportation case 12. In a state of opening the above partition door 44, it becomes possible to carry in/out the rack 36 to/from the container housing room 41. On the other hand, in a state of closing the above partition door 44, an entrance of the container housing room 41 is sealed and is blocked from the outside atmosphere.

Furthermore, inside the container housing room 41, a gas analyzing sensor 46 detecting the composition of the atmosphere gas in the container housing room 41 and a contact-type container detecting switch 47 detecting presence or absence of the rack 36 in the container housing room 41 are arranged. The door driver 45, the gas analyzing sensor 46, and the container detecting switch 47 are connected respectively to the case microcomputer 48 for control built in the container transportation case 12.

Moreover, in the neighborhood of the opening of the case body 40, an electrical contact 49 connected to the above case microcomputer 48 is provided. Then, in an attaching state of the container transportation case 12, the electrical contact 49 on the side of the container transportation case 12 comes into contact with the electrical contact 27 on the side of the incubator 11, and thereby an electrical connection between both is established (the electrical contact 49 is shown only in FIG. 3).

Furthermore, on the upper surface of the case body 40, a gas inlet 50 and a gas outlet 51 are provided to adjust the composition of the atmosphere gas of the container housing room 41. The gas inlet 50 and the gas outlet 51 can be connected to the piping from the gas adjusting unit 18 in an attaching state of the container transportation case 12. Therefore, the gas adjusting unit 18 of the incubator 11 can adjust the composition of the atmosphere gas in the container housing room 41 via the gas inlet 50 and the gas outlet 51.

Further, on a rear side (an opposite side of the opening) of the case body 40, an operation opening 53 is opened to which a flexible glove 52 is mounted. This enables a user to carry out various operations (for example, opening and closing of a lid of the culture container 32 and the like) while confirming a state of the container housing room 41 by inserting a hand into the glove 52 from the operation opening 53 while staying outside.

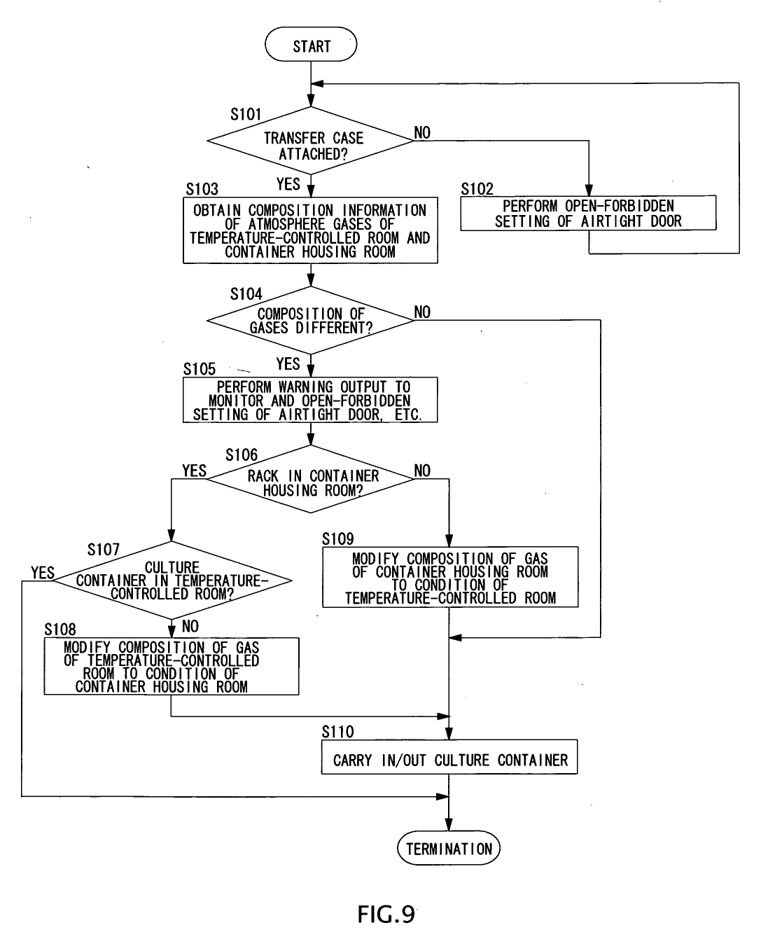

Next, with reference to FIG. 9, regarding the culture treating device system of the present embodiment, a description will be given of a behavioral example of carrying in/out of the culture container 32 by the container transportation case 12. In an initial state of the processing in FIG. 9, the description is given of the premise that the container transportation case 12 is in a non-attaching state to the incubator 11.

Step S101: The controller 37 determines whether or not the container transportation case 12 is attached to the carrying in/out opening 23. As one example, the controller 37 transmits a connection signal to the case microcomputer 48 via the electrical contacts 27 and 49 and also determines that the container transportation case 12 is attached to the carrying in/out opening 23 when receiving a response signal from the case microcomputer 48. When the requirements are satisfied (YES side), the procedure goes on to S101. Meanwhile, when the requirements are not satisfied (NO side), the procedure goes on to S102.

Step S102: In this case, the controller 37 sets the airtight door 24 to be in an open-forbidden state and locks a behavior of the door driver 25 in a state of the airtight door 24 being closed. When opening the airtight door 24 in this setting state, a necessity arises for a user to carry out a particular operation from the operation panel 22 to the controller 37. This reduces a possibility that a user carelessly opens the carrying in/out opening 23. After that, the controller 37 goes back to S101 and repeats the above behaviors.

Step S103: The controller 37 obtains composition information of the atmosphere gas in the temperature-controlled room 16 from the gas analyzing sensor 17. The controller 37 obtains composition information of the atmosphere gas in the container housing room 41 via the electrical contacts 27 and 49 and the case microcomputer 48 from the gas analyzing sensor 46 on the side of the container transportation case 12.

Step S104: The controller 37 determines whether or not the composition of the atmosphere gases in the rooms is different between the temperature-controlled room 16 and the container housing room 41. When both atmosphere gases have the different composition (YES side), the procedure goes on to S105. On the other hand, when the compositions of both atmosphere gases are in the same condition (YES side), the procedure goes on to S110.

Step S105: The controller 37 outputs a warning on the monitor 21 showing that the atmosphere gases of the temperature-controlled room 16 and the container housing room 41 have different compositions. Then, the controller 37 sets the airtight door 24 to be in an open-forbidden state and locks the behavior of the door driver 25 in a state of the airtight door 24 being closed (since the open-forbidden state of the airtight door 24 is similar to S101, a repetitive description is omitted).

Step S106: The controller 37 determines whether or not the rack 36 exists that houses the culture containers 32 in the container housing room 41 of the container transportation case 12 based on output information of the container detecting switch 47 obtained via the electrical contacts 27 and 49 and the case microcomputer 48. When the requirements are satisfied (YES side), the procedure goes on to S107. On the other hand, when the requirements are not satisfied (NO side), the procedure goes on to S109.

Step S107: The controller 37 determines whether or not the culture container 32 exists in the temperature-controlled room 16 of the incubator 11 with reference to the registration information of the culture container 32 memorized in the memory unit 38.

When the requirements are satisfied (YES side), that falls under a condition that respective living body samples exist in the temperature-controlled room 16 and the container housing room 41 and the culture environments of both differ. In this case, when modifying the culture environment to either one of them, there is a high possibility of affecting the other. Accordingly, the controller 37 on the YES side in S107 does not adjust the composition of the atmosphere gases of the temperature-controlled room 16 and the container housing room 41 and terminates a series of processing. On the other hand, when the requirements are not satisfied (NO side), the procedure goes on to S108.

Step S108: This case falls under a condition that a living body sample does not exist in the temperature-controlled room 16, while a living body sample exists which is cultured in a predetermined atmosphere gas-environment in the temperature-controlled room 41. That is, the case of S108 corresponds to a case when a living body sample is first carried in the temperature-controlled room 16 of the incubator 11 using, for example, the container transportation case 12, or the like. In this case, it is preferable that the culture environment on the side of the temperature-controlled room 16 is made closer to the environment of the container housing room 41.

Therefore, the controller 37 in S108 causes the gas adjusting unit 18 to work to thereby carry out control of modifying the composition of the atmosphere gas on the side of the temperature-controlled room 16 to the condition of the container housing room 41. Then, after the compositions of both atmosphere gases become in the same condition, the controller 37 lets the process go on to S110.

Step S109: This case falls under a condition that a living body sample does not exist in the container housing room 41, while a living body sample exists which is cultured in a predetermined atmosphere gas-environment in the temperature-controlled room 16. That is, the case of S109 corresponds to a case when transporting a living body sample cultured in, for example, the temperature-controlled room 16 by putting it in the vacant container transportation case 12, or the like. In this case, it is preferable that the culture environment on the side of the container housing room 41 is made closer to the environment of the temperature-controlled room 16.

Therefore, the controller 37 in S109 causes the gas adjusting unit 18 to work to thereby carry out control of modifying the composition of the atmosphere gas on the side of the container housing room 41 to the condition of the temperature-controlled room 16. Nonetheless, for S109, a necessity arises for a user to connect the piping from the gas adjusting unit 18 in advance to the gas inlet 50 and the gas outlet 51 of the container transportation case 12. Then, after the compositions of both atmosphere gases become in the same condition, the controller 37 lets the process go on to S110.

Step S110: The controller 37 carries in/out the culture container 32 between the container transportation case 12 and the incubator 11.

Specifically, the controller 37 opens the carrying in/out opening 23 by releasing the open-forbidden setting of the airtight door 24 and also causing the door driver 25 to work to thereby slide the airtight door 24. Furthermore, the controller 37 opens the partition door 44 of the container housing room 41 by driving the door driver 45 on the side of the container transportation case 12 via the electrical contacts 27 and 49 and the case microcomputer 48. This allows carrying in/out the culture container 32 between the container housing room 41 and the carrying in/out opening 23, in which the compositions of the atmosphere gases are in the same condition as each other.

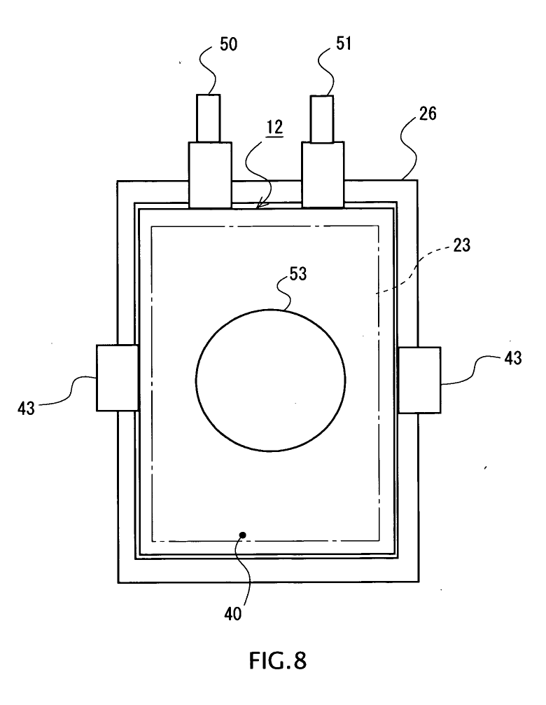

In addition, in a state of attaching the container transportation case 12 to the incubator 11, the entire carrying in/out opening 23 is covered with the container transportation case 12, and also a joint portion of the container transportation case 12 and the incubator 11 is under airtight conditions by the seal ring 42 (refer to FIG. 8). Therefore, in S110, the culture container 32 can be carried in/out in an environment that is blocked from the outside atmosphere.

Here, when the culture container 32 is housed in the container transportation case 12 together with the rack 36, the controller 37 causes the container carrying in/out unit 31 to work to thereby carry the culture container 32 into the temperature-controlled room 16 from the container housing room 41. Subsequently, the controller 37 closes the airtight door 24 and the partition door 44, and then terminates a series of behaviors.

On the other hand, when the culture container 32 is in the container carrying in/out unit 31 of the temperature-controlled room 16 together with the rack 36, the controller 37 causes the container carrying in/out unit 31 to work to thereby carry the culture container 32 out to the container housing room 41 from the temperature-controlled room 16. After that, the controller 37 closes the airtight door 24 and the partition door 44, and then terminates a series of behaviors. In addition, in this case, the composition of the atmosphere gas of the container housing room 41 is in the same condition as that of the temperature-controlled room 16, and also the entrance side of the container housing room 41 is blocked from an ambient air by the partition door 44. Therefore, a user can transport the living body sample while maintaining the preferred environmental conditions by using the container transportation case 12. This is the end of the description of the flowchart in FIG. 9.

### Supplementary Notes to Embodiment

(1) In the culture treating device system of the above embodiment, a description is given of a combination of the incubator 11 having the temperature-controlled room 16 and the container transportation case 12. However, the culture treating device of the present invention may also be, for example, a clean bench to carry out a culture operation of a living body sample or the like.

(2) Regarding the container transportation case 12 of the above embodiment, although an example of arranging the operation opening 53 with the glove 52 in the back of the container housing room 41 is described, operation openings 53 with gloves 52 may also be formed, for example, on both sides of the container housing room 41, respectively (illustration of this case is omitted).

(3) Although the description is given of an example of electrically opening and closing both the airtight door 24 of the incubator 11 and the partition door 44 of the container transportation case 12 in the above embodiment, the doors may also be those that may be opened and closed manually by a user as long as the controller 37 can lock them.

The many features and advantages of the embodiments are apparent from the detailed specification and, thus, it is intended by the appended claimed to cover all such features and advantages of the embodiments that fall within the true spirit and scope thereof. Further, since numerous modifications and changes will readily occur to those skilled in the art, it is not desired to limit the inventive embodiments to the exact construction and operation illustrated and described, and accordingly all suitable modifications and equivalents may be resorted to, falling within the scope thereof.

The many features and advantages of the embodiment are apparent from the detailed specification and, thus, it is intended by the appended claims to cover all such features and advantages of the embodiment that fall within the true spirit and scope thereof. Further, since numerous modifications and changes will readily occur to those skilled in the art, it is not desired to limit the inventive embodiment to the exact construction and operation illustrated and described, and accordingly all suitable modifications and equivalents may be resorted to, falling within the scope thereof.

What is claimed is:

1. A container transportation case for carrying in/out a culture container to/from a chamber, relative to a culture treating device having the chamber capable of adjusting composition of an atmosphere gas therein to a predetermined condition, the culture container culturing a living body sample, the container transportation case comprising:
a case body having a container housing room in which the culture container can be arranged, the container housing room being in communication with outside by an opening part through which the culture container can pass;
a joint unit capable of being attached/removed to/from a carrying-in/out opening of the culture treating device in communication with the chamber and forming a space isolated from an outside atmosphere by joining the container housing room and the carrying-in/out opening in an attaching state to the culture treating device; and
an airtight member blocking the container housing room from the outside atmosphere by closing the opening part.

2. The container transportation case according to claim 1, further comprising a gas inlet and a gas outlet adjusting composition of an atmosphere gas of the container housing room.

3. The container transportation case according to claim 1, wherein an operation opening having a flexible glove mounted thereon is formed on at least one surface of the case body which configures the container housing room.

4. A culture treating device to which the container transportation case according to claim 1 can be attached, the container transportation case further including a first analyzing sensor which generates first information indicating composition of an atmosphere gas in the container housing room and a case-side contact which outputs the first information to the culture treating device, the culture treating device comprising:
a chamber capable of adjusting composition of an atmosphere gas therein to a predetermined condition;
a carrying-in/out opening being in communication with the chamber;
an airtight door capable of sealing the carrying-in/out opening;
a second analyzing sensor generating second information which shows the composition of the atmosphere gas in the chamber;
a device-side contact obtaining the first information from the case-side contact when the container transportation case is attached; and
a controlling unit performing at least one of an open-forbidden setting of the airtight door and an output process of a warning when determining that the atmosphere gases of the container housing room and the chamber have different composition based on the first information and the second information.

5. The culture treating device according to claim 4, further comprising a gas adjusting unit which adjusts the composition of either one of the atmosphere gases of the container housing room and the chamber when the controlling unit determines that the atmosphere gases of the container housing room and the chamber have different composition.

6. The culture treating device according to claim 5, wherein
the container transportation case further includes a culture container detecting unit which detects presence or absence of the culture container in the container housing room, and
the gas adjusting unit adjusts the composition of the atmosphere gas of the chamber in a state of the culture container being in the container housing room, and adjusts the composition of the atmosphere gas in the container housing room in a state of the culture container not being in the container housing room.

7. A culture treating device to which the container transportation case according to claim 1 can be attached, comprising:
a chamber capable of adjusting composition of an atmosphere gas therein to a predetermined condition;
a carrying-in/out opening being in communication with the chamber;
an airtight door capable of sealing the carrying-in/out opening;
an attached/removed detecting unit detecting an attaching state and a non-attaching state of the container transportation case; and
a controlling unit carrying out an open-forbidden setting of the airtight door when the container transportation case is not attached.
